(19) United States
(12) Patent Application Publication
(10) Pub. No.: US 2001/0007872 A1
(43) Pub. Date: Jul. 12, 2001

(54) METHOD OF TREATING ACUTE, CHRONIC AND/OR NEUROPATHIC PAIN

(76) Inventors: FRANK S. MENNITI, MYSTIC, CT (US); BERTRAND L. CHENARD, WATERFORD, CT (US); MARIO D. SALTARELLI, MYSTIC, CT (US); JONATHON M. PARKER, SALEM, CT (US)

Correspondence Address: PFIZER INC, 235 E 42ND STREET, NEW YORK, NY, 10017, US

(21) Appl. No.: 09/397,891

(22) Filed: Sep. 17, 1999

Related U.S. Application Data
(60) Provisional application No. 60/102,630, filed on Oct. 1, 1998.

(51) Int. Cl. A61K 31/55; A61K 31/44; A61K 31/47; A61K 31/445

(52) U.S. Cl. 514/278; 514/327; 514/216; 514/315

(57) ABSTRACT

This invention relates to a method of treating acute, chronic and/or neuropathic pain in which a mammal suffering from acute, chronic and/or neuropathic pain is treated with an effective amount of an NR2B selective NMDA antagonist having a ratio of NR2B receptor activity to α₁-adrenergic receptor activity of at least about 3:1.

This application is based on U.S. Provisional Patent Application Ser. No. 60/102,630, filed Oct. 1, 1998.

### BACKGROUND OF THE INVENTION

This invention relates to the field of treating acute, chronic and/or neuropathic pain. Specifically, this invention relates to a method of treating mammals, including humans, suffering from acute, chronic and/or neuropathic pain, which method comprises administering to mammals in need of such treatment an amount of compound which inhibits selectively N-methyl-D-aspartate (hereinafter NMDA) receptors containing the NR2B subunit (hereinafter NR2B selective antagonist). More specifically, the invention relates to a method of treating acute, chronic and/or neuropathic pain with a decrease in the severity of unwanted side-effects, particularly a method of treating acute, chronic and/or neuropathic pain without significantly affecting blood pressure.

Glutamate and aspartate play dual roles in the central nervous system as essential amino acids and the principal excitatory neurotransmitters (hereinafter referred to as excitatory amino acids or EAAs). There are at least four classes of EAA receptors, specifically NMDA, AMPA (2-amino-3-(methyl-3-hydroxyisoxazol-4-yl)propanoic acid), kainate and metabotropic. These EAA receptors mediate a wide range of signaling events that impact all physiological brain functions. The focus of the present invention is the role of NMDA receptors containing the NR2B subunit in pain perception and the analgesic activity of selective NR2B receptor antagonists.

NMDA receptor inhibition decreases pain perception (Wong, C. S., Cherng, C. H. and Ho, S. T., Clinical Applications of Excitatory Amino Acid Antagonists in Pain Management [Review][48 references] Acta Anaesthesiologica.Sinica; 33, 227-232 (1995)). In animal models of pain states, NMDA receptor antagonists inhibit acute pain perception. These compounds also inhibit pain sensitization processes in which the perception of the painfulness of a given stimulus is increased without change in stimulus intensity. In humans, NMDA receptor antagonists have also been found to decrease both acute pain perception and sensitization. However, while NMDA receptor inhibition has therapeutic utility in the treatment of pain, there are significant liabilities to many available NMDA receptor antagonists. Specifically, many NMDA receptor antagonists that have been tested in humans can cause potentially serious side effects including memory disruption, induction of psychotic-like symptoms and disruption of cardiovascular function. These side effects in humans are thought to be related to similar phenomena observed in animals, including induction of locomotor hyperactivity and stereotypy and, in rodents, a hypermetabolic state in discreet areas of the brain that can result in apparent neuronal damage. These phenomena are indicated by the presence of neuronal vacuoles in formalin-fixed brain sections. Thus, a significant advance would be to discover NMDA receptor antagonists that decrease pain perception and sensitization at doses producing a lesser degree of the aforementioned side effects. One approach to developing analgesic NMDA receptor antagonists with a better ratio of therapeutic effect to side effect is to target subtypes of the NMDA receptor specifically involved in pain perception.

The NMDA receptor is an ion channel gated by synaptically released EAA in the presence of coagonist glycine and concomitant depolarization (Mayer, M. L. and Westbrook, G. L., The Physiology of Excitatory Amino Acids in the Vertebrate Nervous System, Progress in Neurobiology, 28, 197-276 (1987)). Thus, NMDA receptor activity may be attenuated by blockade of the glutamate binding site, the glycine coagonist binding site or the receptor-associated ion channel. The NMDA receptor is composed of multiple protein subunits (Seeburg, P. H., The Molecular Biology of Mammalian Glutamate Receptor Channels, Trends in Neurosci., 16, 359-365 (1993)). Five subunits have been cloned to date, NR1 and NR2A through D. Expression studies indicate the functional receptor is composed of at least one NR1 subunit and one or more of the NR2 subunits. In the adult mammalian brain, the NR1 and NR2A subunits are widely expressed. In contrast, NR2B subunit expression is mostly localized in forebrain regions including cortex, hippocampus and striatum whereas the NR2C subunit is expressed in the cerebellum and the NR2D subunit is restricted to the midbrain region. Thus, different NMDA receptors subtypes are formed from different combinations of receptor subunits that are differentially expressed throughout the central nervous system. Compounds that inhibit NMDA receptor activity by interacting at the glutamate, glycine, and receptor-associated ion channel have little (<10-fold) selectivity across the different receptor subtypes. That is, such compounds inhibit NMDA receptors with potencies within a 10-fold range regardless of the subunit combination.

NMDA receptors containing the NR2B subunit have a unique site to which compounds may bind to specifically inhibit this subtype (Menniti, F. S. and Chenard, B. L., Antagonists Selective for NMDA Receptors Containing the NR2B Subunit, Current Pharmaceutical Design, 1999, 5:381-404)). A number of compounds have been found to act as antagonists that target the NR2B subunits of the NMDA receptors. The first compound identified to display significant affinity for this NR2B-specific site was ifenprodil. Ifenprodil is both more potent and efficacious for blockade of ion current through NMDA receptors comprised of NR1/NR2B subunits compared to NR1/NR2A, NR2C, or NR2D subunits. Ifenprodil is a well known α₁-adrenoceptor antagonist exhibiting NMDA receptor antagonist activity via interaction with the polyamine modulatory site . (Carter et al. J. Pharmacol. Exp. Ther., 235, 475-482 (1990)).

Ifenprodil and related compounds have been demonstrated in animal models of pain perception to produce significant analgesic activity (Bernardi, M., Bertolini, A., Szczawinska, K, And Genedani, S., Blockade of the Polyamine Site of NMDA Receptors Produces Antinociception and Enhances the Effect of Morphine, in Mice, European Journal of Pharmacology, 298, 51-55, (1996); Taniguchi, K., Shinjo, K., Mizutani, M., Shimada, K., Ishikawa, T., Menniti, F. S. and Nagahisa, A, Antinociceptive Activity of CP-101,606, an NMDA Receptor NR2B Subunit Antagonist, British Joumal of Pharmacology, 122, 809-812 (1997)). These data indicate that NMDA receptors containing the NR2B subunit play a role in mediating pain perception. Furthermore, these compounds do not produce locomotor hyperactivity or neuronal vacuoles in rodents and are well tolerated in humans at doses expected to produce analgesic activity (Menniti, F. S. and Chenard, B. L., Antagonists Selective for NMDA Receptors Containing the NR2B Subunit, Current Pharmaceutical Design, 1999, 5:381-404)). These data further indicate that the selective inhibition of NMDA receptors containing the NR2B subunit can produce analgesic activity with a greater ratio of therapeutic effect to side effect than can be realized with NMDA receptor antagonists that lack selectivity for the NR2B receptor subtype.

Although ifenprodil has selectivity for the NR2B subtype of NMDA receptor, this compound also interacts with and inhibits a number of other receptors and ion channels. In particular, ifenprodil inhibits the α₁ adrenergic receptor with an affinity similar to that at which the compound inhibits NR2B subtype NMDA receptors. Inhibition of α₁ adrenergic receptors is related to particular structural features of ifenprodil and related molecules (Chenard, B. L., Shalaby, I. A., Koe, B. K., Ronau, R. T., Butler, T. W., Prochniak, M. A., Schmidt, A. W. and Fox, C. B.; Separation of α₁ Adrenergic and N-methyl, D-aspartate Antagonist Activity in a Series of Ifenprodil Compounds, J. Med. Chem., 34, 3085-3090 (1991)). Furthermore, inhibition of α₁ adrenergic receptors causes a reduction in blood pressure. This can be a serious complication to the therapeutic use of ifenprodil and similar compounds. Thus, a further significant advance would be to discover NR2B selective antagonists that decrease pain perception and sensitization at doses that do not significantly inhibit α₁ adrenergic receptors and cause little or no change in blood pressure.

Compounds of formula I are described in U.S. Pat. Nos. 5,185,343, 5,272,160, 5,338,754, and 5,356,905 (which issued, respectively, on Feb. 9, 1993; Dec. 21, 1993; Aug. 16, 1994; and Oct. 18, 1994); U.S. patent applications Ser. No. 08/292,651 (filed Aug. 18, 1994), Ser. No. 08/189,479 (filed Jan. 31, 1994) and Ser. No. 09/011,426 (filed Jun. 20, 1996); PCT International Application No. PCT/IB95/00398 which designates the United States (filed May 26, 1995) (corresponding to WO 96/37222); PCT International Application No. PCT/IB95/00380 which designates the United States (filed May 18, 1995) (corresponding to WO 96/06081). All of the foregoing patents, United States patent applications and PCT international application are herein incorporated by reference in their entirety.

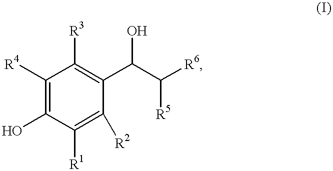

The compounds of formula I are NMDA receptor antagonists. Non-selective antagonists of neurotransmission at NMDA receptors are useful therapeutic agents for the treatment of neurological disorders. (J. Lehman, The NMDA Receptor, Drugs of the Future, 14(11), 1059 (1989)). U.S. Pat. No. 4,902,695 is directed to series of competitive NMDA antagonists useful for the treatment of neurological disorders, including epilepsy, stroke, anxiety, cerebral ischemia, muscular spasms, and neurodegenerative disorders such as Alzheimer's disease and Huntington's disease. NMDA antagonists, in general, have also been reported to be effective for treating migraine (Canadian J. of Neurological Science, 19(4), 487 (1992)); drug addiction (Science, 251, 85 (1991)); and neuro-psychotic disorders related to AIDS (PIPS, 11, 1 (1990)).

The present invention is directed to the discovery that NMDA receptor antagonists selective for receptors containing the NR2B subunit are capable of providing analgesic activity while simultaneously causing a lessened degree of undesirable side effects compared to nonselective NMDA receptor antagonists. The importance of NR2B selectivity, and the advantage of using an NR2B selective NMDA receptor antagonist over a non-selective NMDA receptor antagonist, such as MK801 ((+) 5-methyl-10,11-dihydro-5H-dibenzo[a,d]cyclohepten-5,10-imine), CGS-19,755 (cis-4-(phosphonomethyl)-(2-piperidinecarboxylic acid)), CNS-1102 (N-(3-(ethylphenyl)-N-methyl-N′-1-naphthalenyl-guanidine) and memantine (3,5-dimethyl-tricyclo[ 3.3.1.13.7]decan-1-amine), was not previously recognized. The present invention teaches that the NR2B-containing NMDA receptors are important sites of action for the non-selective agents and adverse effects exhibited upon administration of certain non-selective NMDA receptor antagonists are not observed, or are significantly reduced, when using NR2B selective NMDA receptor antagonists. Thus, the present invention provides a preferential treatment for acute, chronic and/or neuropathic pain, with lower occurrence and/or lessened severity of adverse side effects in which pain is treated by administering an NR2B selective NMDA receptor antagonist.

Further, structural features of many NR2B selective receptor antagonists result in concurrent and equipotent inhibition of α₁ adrenergic receptor with a resultant effect on blood pressure. The therapeutic advantage of the NR2B selective receptor antagonists is realized specifically only with compounds lacking significant inhibitory activity at α₁ adrenergic receptors, and therefore, do not induce potential cardiovascular side effects via this mechanism.

### SUMMARY OF THE INVENTION

The present invention is directed to a method of treating acute, chronic and/or neuropathic pain in a mammal, which method comprises administering to a mammal in need of such treatment an amount effective to attenuate said pain of an NR2B selective N-methyl-D-aspartate (NMDA) receptor antagonist.

In one preferred embodiment, said NR2B selective NMDA receptor antagonist is a compound that has ratio of NR2B receptor selectivity to α₁ adrenergic receptor selectivity of at least about 3:1, preferably of at least 5:1.

A preferred method within the scope of this invention is a method as described in the preceding paragraph wherein said NR2B subtype selective NMDA receptor antagonist is a compound of the formula

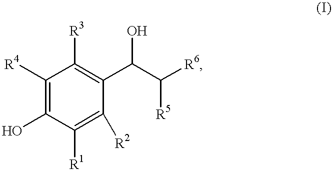

or a pharmaceutically acceptable acid addition salt thereof, wherein:

(a) R² and R⁵ are taken separately and R¹, R², R³ and R⁴ are each independently hydrogen, (C₁-C₆) alkyl, halo, CF₃, OH or OR⁷ and R⁵ is methyl or ethyl; or

(b) R² and R⁵ are taken together and are

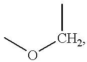

forming a chroman-4-ol ring, and R¹, R³ and R⁴ are each independently hydrogen, (C₁-C₆) alkyl, halo, CF₃, OH or OR⁷;

R⁶ is

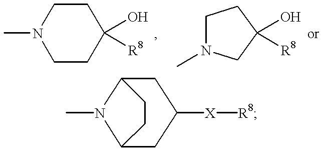

R⁷ is methyl, ethyl, isopropyl or n-propyl;

R⁸ is phenyl optionally substituted with up to three substituents independently selected from (C₁-C₆) alkyl, halo and CF₃;

X is O, S or (CH₂)ₙ; and

n is 0, 1, 2, or 3.

Preferred compounds for use in the present invention include those of formula I wherein R² and R⁵ are taken separately; R² and R³ are hydrogen; R⁶ is

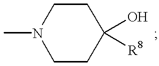

and R⁸ is phenyl, 4-halophenyl or 4-trifluoromethylphenyl. Within this group, more specific preferred compounds are those wherein R⁵ is methyl having a 1S*, 2S* relative stereochemistry:

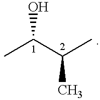

Other preferred compounds to be used in accord with the present invention compounds include those of formula I wherein R² and R⁵ are taken together and are

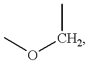

forming a chroman-4-ol ring. Within this group, preferred compounds also include those wherein the C-3 and C-4 positions of said chroman-4-ol ring have a 3R*, 4S* relative stereochemistry:

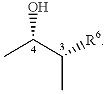

Within this group, preferred compounds also include those wherein R⁶ is

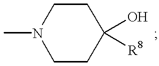

and R⁸ is phenyl or 4-halophenyl.

Three compounds within the scope of this invention as described in the preceding paragraph are particularly preferred. These compounds are (+)-(1S, 2S)-1-(4-hydroxy-phenyl)-2-(4-hydroxy-4-phenylpiperidino)-1-yl)-1-propanol; (1S, 2S)-1-(4-hydroxy-3-methoxyphenyl)-2-(4-hydroxy-4-phenylpiperidino)-1-propanol; and (3R,4S)-3-(4-(4-fluorophenyl)-4-hydroxypiperidin-1-yl)-chroman-4,7-diol.

### DETAILED DESCRIPTION OF THE INVENTION

The compounds of formula I are readily prepared. The compounds of formula I wherein R² and R⁵ are taken together forming a chroman-4-ol ring and R¹, R³, and R⁴ are hydrogen, can be prepared by one or more of the synthetic methods described and referred to in U.S. Pat. No. 5,356,905. The compounds of formula I wherein R² and R⁵ are taken separately and R¹, R², R³ and R⁴ are hydrogen can be prepared by one or more of the synthetic methods described and referred to in U.S. Pat. Nos. 5,185,343, 5,272,160, and 5,338,754. The compounds of formula I can also be prepared by one or more of the synthetic methods described and referred to in U.S. patent application Ser. Nos. 08/292,651, 08/189,479 and 09/011,426; PCT International Application No. PCT/IB95/00398 which designates the United States (filed May 26, 1995) (corresponding to WO 96/37222); PCT International Application No. PCT/IB95/00380 which designates the United States (filed May 18, 1995) (corresponding to WO 96/06081). The foregoing United States patents, United States applications and PCT international application are referred to above.

A preferred compound, (1S,2S)-1-(4-hydroxyphenyl)-2-(4-hydroxy-4-phenylpiperidin-1-yl)-1-propanol ((1S,2S) free base), and its tartrate salt, can be prepared as described in U.S. Pat. No. 5,272,160, referred to above. The resolution of racemic 1-(4-hydroxyphenyl)-2-(4-hydroxy-4-phenylpiperidin-1-yl)-1-propanol to form the (1S,2S) free base and the corresponding (1R,2R) enantiomer can be carried out as described in U.S. patent application Ser. No. 09/011,426, referred to above, and as exemplified in Example 1 below.

The anhydrous mesylate of the (1S,2S) free base can be prepared as described in U.S. Pat. No. 5,272,160, referred to above. The anhydrous mesylate of the (1S,2S) free base, when equilibrated in an 81% relative humidity environment, will convert to the mesylate salt trihydrate of the (1S,2S) enantiomer.

The mesylate salt trihydrate of (1S,2S)-1-(4-hydroxyphenyl)-2-(4-hydroxy-4-phenylpiperidin-1-yl)-1-propanol can be prepared from the (1S,2S) free base as described in United States provisional patent application entitled “(1S,2S)-1-(4-Hydroxyphenyl)-2-(4-Hydroxy-4-Phenylpiperidin-1-yl)-1-Propanol Methanesulfonate Trihydrate”, referred to above. In this method, (1S,2S) free base is dissolved in water at 30° C. To this solution is added at least 1 equivalent of methane sulfonic acid and the resulting mixture is warmed to 60-65° C. The warm solution can be filtered to render it particulate free. The solution is concentrated to approximately 40% of the initial volume, cooled below 10° C., isolated by filtration and dried to a water content (measured Karl Fischer titration) of approximately 11.3%. The resulting crystalline mesylate salt trihydrate can be further purified by recrystallization.

Another preferred compound, (3R,4S)-3-[4-(4-fluorophenyl)-4-hydroxy-piperidin-1-yl] -chroman-4,7-diol ((3R,4S) chromanol), can be prepared as described in U.S. Pat. No. 5,356,905, U.S. patent application Ser. No. 08/189,479, and United States provisional patent application entitled “Process For The Resolution Of Cis-Racemic 7-Benzyloxy-3-[4-(4-Fluorophenyl)4-Hydroxy-Piperidin-1-yl]-Chroman-4-ol Dibenzoyl-D-Tartrate”, all three of which are referred to above. The starting materials and reagents required for the synthesis of the (3R,4S) chromanol are readily available, either commercially, according to synthetic methods disclosed in the literature, or by synthetic methods exemplified in the description provided below.

The (3R,4S) chromanol can be prepared by fractional crystallization of the L-proline ester of racemic cis-7-benzyloxy-3-[4-(4-fluorophenyl)-4-hydroxy-piperidin-1-yl]-chroman4-ol, as described in U.S. patent application Ser. No. 08/189,479, referred to above. In a preferred method, the resolution method described in United States provisional patent application entitled “Process For The Resolution Of Cis-Racemic 7-Benzyloxy-3-[4-(4-Fluorophenyl)-4-Hydroxy-Piperidin-1-yl]-Chroman-4-ol Dibenzoyl-D-Tartrate”, referred to above, and as exemplified in Example 3. In this method, the parent chromanol is prepared by dissolving racemic cis-7-benzyloxy-3-[4-(4-fluorophenyl)-4-hydroxy-piperidin-1-yl]-chroman-4-ol with an equal molar amount of dibenzoyl-D-tartaric acid in boiling aqueous ethanol. Racemic cis-7-benzyloxy-3-[4-(4-fluorophenyl)-4-hydroxy-piperidin-1-yl]-chroman-4-ol is prepared as described in U.S. patent application Ser. No. 08/189,479, referred to above. The concentration of aqueous ethanol is not critical and may be varied between 75% and 95% ethanol (ETOH). A concentration of 9:1/ETOH:H₂O has been found to be effective and is preferred. A sufficient amount of the aqueous ethanol solvent to dissolve the racemic compound is required. This amount has been found to be about 17 ml per gram of racemic compound.

Upon stirring while heating under reflux, the racemic compound dissolves to form a hazy solution which is allowed to cool with stirring whereupon the (+) isomer, (3R,4S)-7-benzyloxy-3-[4-(4-fluorophenyl)-4-hydroxy-piperidin-yl]-chroman4-ol dibenzoyl-D-tartrate, precipitates and may be collected by filtration and washed with aqueous ethanol. This is the tartrate salt of the (3R,4S) chromanol. The lactate and mandelate salts of the (3R,4S) chromanol are prepared in an analogous manner. This initial product is of about 90% optical purity. If a higher purity is desired, the product may be heated again with aqueous ethanol, cooled and the product collected and washed. Two such treatments were found to yield the (+) isomer of 99.4% optical purity in an overall yield of 74%. This procedure is preferred over the procedure described in U.S. patent application Ser. No. 08/189,479, referred to above, in that it avoids a reduction step with lithium aluminum hydride and is therefore more suitable for bulk operations. This procedure also produces a significantly higher yield of the desired product.

The above described (+) isomer can be converted to (3R,4S)-3-[4-(4-fluorophenyl)-4-hydroxy-piperidin-1-yl]-chroman-4,7-diol by standard procedures. For example, treatment with dilute base can be used to free the piperidinyl base and subsequent hydrogeneration removes the 7-benzyl group to yield the (3R,4S) chromanol.

In general, the pharmaceutically acceptable acid addition salts of the compounds of formula I can readily be prepared by reacting the base forms with the appropriate acid. When the salt is of a monobasic acid (e.g., the hydrochloride, the hydrobromide, the p-toluenesulfonate, the acetate), the hydrogen form of a dibasic acid (e.g., the dihydrogen phosphate, the citrate), at least one molar equivalent and usually a molar excess of the acid is employed. However, when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

It is to be understood that other NR2B subtype selective NMDA receptor antagonists, are within the scope of the method of this invention. NR2B selectivity can be determined, for example, by the following assay.

Selectivity of compounds for the NR2B-subunit containing NMDA receptor is defined as an affinity for the racemic [³H](+)-(1S, 2S)-1-(4-hydroxy-phenyl)-2-(4-hydroxy-4-phenylpiperidino)-1-propanol binding site in forebrain of rats, as described in Chenard and Menniti (Antagonists Selective for NMDA receptors containing the NR2B Subunit, Current Pharmaceutical Design, 1999, 5:381-404). This affinity is assessed in a radioligand binding assay as described below. Selective compounds are those which displace specific binding of racemic [³H]CP-101,606 from rat forebrain membranes with an IC₅₀≦5 μM.

The binding of racemic [³H] (+)-(1S, 2S)-1-(4-hydroxy-phenyl)-2-(4-hydroxy-4-phenylpiperidino)-1-propanol to rat forebrain membranes is measured as described by Menniti et al. (CP-101,606, a potent neuroprotectant selective for forebrain neurons, European Journal of Pharmacology, 1997, 331:117-126). Forebrains of adult male CD rats are homogenized in 0.32M sucrose at 4° C. The crude nuclear pellet is removed by centrifugation at 1,000×g for 10 min., and the supernatant centrifuged at 17,000×g for 25 min. The resulting pellet is resuspended in 5 mM Tris acetate pH 7.4 at 4° C. for 10 min. to lyse cellular particles and again centrifuged at 17,000×g. The resulting pellet is washed twice in Tris acetate, resuspended at 10 mg protein/ml and stored at −20° C. until use.

For binding assays, membranes are thawed, homogenized, and diluted to 0.5 mg protein/ml with 50 mM Tris HCl, pH 7.4. Compounds under study are added at various concentrations followed by racemic [³H] (+)-(1S, 2S)-1-(4-hydroxy-phenyl)-2-(4-hydroxy-4-phenylpiperidino)-1-propanol (specific activity 42.8 Ci/mmol, 5 nM final concentration) Following incubation for 20 min at 30° C. in a shaking water bath, samples are filtered onto Whatman GFB glass fiber filters using a MB-48R Cell Harvester (Brandel Research and Development Laboratories, Gaithersburg Md.). Filters are washed for 10 s with ice cold Tris HCl buffer and the radioactivity trapped on the filter quantified by liquid scintillation spectroscopy. Nonspecific binding is determined in parallel incubations containing 100 μM racemic (+)-(1S, 2S)-1-(4-hydroxy-phenyl)-2-(4-hydroxy-4-phenylpiperidino)-1-propanol. Specific binding is defined as total binding minus nonspecific binding.

Compounds claimed in this patent have selectivity for NR2B subunit-containing NMDA receptors over α₁-adrengergic receptors. Affinity for the NR2B subunit containing NMDA receptor is measured as the IC₅₀ for displacement of specific binding of racemic [³H] (+)-(1S, 2S)-1-(4-hydroxy-phenyl)-2-(4-hydroxy-4-phenylpiperidino)-1-propanol from rat forebrain membranes (described above). Affinity for the α₁-adrengergic receptor is defined as the IC₅₀ for displacement of specific binding of racemic [³H]prazosin from rat brain membranes, measured as described by Greengrass and Bremner (Binding Characteristics of [³H]prazosin to Rat Brain (α-Adrenergic Receptors, European Journal of Pharmacology, 55, 323-326, (1979)). A compound with a ratio of ([³H]prazosin/[³H] (+)-(1S, 2S)-1-(4-hydroxy-phenyl)-2-(4-hydroxy-4-phenylpiperidino)-1-propanol) affinity greater than three is considered selective.

Forebrains of adult male Sprague Dawley rats are homogenized in 20 volumes of ice cold 50 mM Tris/HCl buffer (pH 7.7). The homogenate is centrifuged at 50,000 X g for 10 min. at 4° C. The pellet is resuspended and centrifuged under identical conditions and the final pellet is resuspended in 80 volumes of 50 mM Tris/HCl (pH 8.0) at 4° C.

For binding assays, compounds under study are added at various concentrations to 500 μg membrane protein in 1 ml of 50 mM Tris/HCl buffer, followed by [³H]prazosin (Amersham, specific activity 33 Ci/mmol, 0.2 nM final concentration). Following incubation for 30 min at 25° C. in a shaking water bath, samples are filtered onto Whatman GFB glass fiber filters using a MB48R Cell Harvester (Brandel Research and Development Laboratories, Gaithersburg Md.). Filters are washed three times for 10 s with ice cold Tris HCl buffer and the radioactivity trapped on the filter quantified by liquid scintillation spectroscopy. Nonspecific binding is determined in parallel incubations containing 100 nM prazosin. Specific binding is defined as total binding minus nonspecific binding.

NR2B selective NMDA receptor antagonists useful in the practice of the invention may also be used in the form of a pharmaceutically acceptable salt. The expression “pharmaceutically-acceptable acid addition salts” is intended to include but not be limited to such salts as the hydrochloride, hydrobromide, sulfate, hydrogen sulfate, phosphate, hydrogen phosphate, dihydrogenphosphate, acetate, succinate, citrate, tartrate, lactate, mandelate, methanesulfonate (mesylate) and p-toluenesulfonate (tosylate) salts. The acid addition salts of the compounds of the present invention are readily prepared by reacting the base forms with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the acid is employed. However when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

The above-described NR2B subunit selective NMDA receptor antagonists can be used to great advantage in the treatment of chronic and/or acute pain conditions in mammals. Such compounds have been found not to cause, or to ameliorate the severity of many side effects encountered with the use of non-selective NMDA receptor antagonist (e.g., MK-801, CGS-19,755, CNS-1102 and memantine). Adverse side effects associated with the use such non-selective NMDA receptor antagonists that are avoided or ameliorated by use of the present treatment methods include reductions in motor coordination (ataxia), sedation, hallucinatory effects or hyperactivity.

Due to the nature of acute and/or chronic pain, the analgesic activity of the NR2B selectivive NMDA receptor antagonist will occur over a wide dose range. For example, the NR2B selective NMDA receptor antagonist, particularly (+)-(1S, 2S)-1-(4-hydroxy-phenyl)-2-(4-hydroxy-4-phenylpiperidino)-1-propanol, may be administered over the dose range of about 0.02 mg to about 10 mg per kilogram of body weight of patient per day (1 to 500 mg/day in a typical human weighing 50 kg). Proper dosages for a given patient can be readily determined by the clinician of ordinary skill in the art of treating acute and/or chronic pain enabled by this disclosure.

The NR2B selective NMDA receptor antagonist useful in the method of the present invention is generally administered in the form of a pharmaceutical composition comprising one or more NR2B selective NMDA receptor antagonists together with a pharmaceutically acceptable carrier or diluent. Such compositions are generally formulated in a conventional manner utilizing solid or liquid vehicles or diluents as appropriate to the mode of administration.

For purposes of oral administration, tablets containing excipients such as sodium citrate, calcium carbonate and dicalcium phosphate may be employed along with various disintegrants such as starch and preferably potato or tapioca starch, alginic acid and certain complex silicates, together with binding agents such as polyvinylpyrrolidone, sucrose, gelatin and acacia. Additionally, lubricating agents such as, but not limited to, magnesium stearate, sodium lauryl sulfate and talc are often very useful for tableting purposes. Solid compositions of a similar type may also be employed as fillers in soft elastic and hard-filled gelatin capsules; preferred materials in this connection also include, by way of example and not of limitation, lactose or milk sugar as well as high molecular weight polyethylene glycols. When aqueous suspensions and/or elixirs are desired for oral administration, the essential active ingredient may be combined with various sweetening or flavoring agents, coloring matter or dyes and, if so desired, emulsifying and/or suspending agents, together with diluents such as water, ethanol, propylene glycol, glycerin and various like combinations thereof.

The term analgesic activity, when used herein and in the appended claims, defines an effect that alleviates or ameliorates the symptomatology of acute and/or chronic pain.

The present invention is illustrated by the following examples, but is not limited to the details thereof.

All nonaqueous reactions were run under nitrogen for convenience and generally to maximize yields. All solvents/diluents were dried according to standard published procedures or purchased in a predried form. All reactions were stirred either magnetically or mechanically. NMR spectra are recorded at 300 MHz and are reported in ppm. The NMR solvent was CDCl₃ unless otherwise specified. IR spectra are reported in cm⁻¹, generally specifying only strong signals.

### EXAMPLE 1

### Enantiomeric (1S, 2S)- and (1 R, 2R)-1-(4-Hydroxy-phenyl)-2-(4-hydroxy-4-phenylpiperidin-1-yl)-1-propanol

(+)-Tartaric acid (300 mg, 2 mmol) was dissolved in 30 mL warm methanol. Racemic 1S*, 2S*-1-(4-hydroxyphenyl)-2-(4-hydroxy-4-phenylpiperidin-1-yl)-1-propanol (655 mg, 2 mmol) was added all at once. With stirring and gentle warming a colorless homogeneous solution was obtained. Upon standing at ambient temperature 24 hours, 319mg (66%) of a fluffy white precipitate was obtained. This product was recrystallized from methanol to give 263 mg of the (+)-tartrate salt of levorotatory title product as a white solid; mp 206.5-207.5° C.; [alpha]_{D}=−36.2°. This salt (115 mg) was added to 50 mL of saturated NaHCO₃. Ethyl acetate (5 mL) was added and the mixture was vigorously stirred 30 minutes. The aqueous phase was repeatedly extracted with ethyl acetate. The organic layers were combined and washed with brine, dried over calcium sulfate, and concentrated. The tan residue was recrystallized from ethyl acetate-hexane to give 32 mg (39%) of white, levorotatory title product; mp 203-204° C.; [alpha]_{D}=−58.4°. Anal. Calc'd. for C₂₀H₂₅NO₃: C, 73 37; H, 7.70; N, 4.28. Found: C, 72.61; H, 7.45; N, 4.21.

The filtrate from the (+)-tartrate salt preparation above was treated with 100 mL saturated aqueous NaHCO₃ and extracted well with ethyl acetate. The combined organic extracts were washed with brine, dried over calcium sulfate and concentrated to give 380 mg of recovered starting material (partially resolved). This material was treated with (−)-tartaric acid (174 mg) in 30 mL of methanol as above. After standing for 24 hours, filtration gave 320 mg (66%) of product which was further recrystallized from methanol to produce 239 mg of the (−)-tartrate salt of dextrorotatory title product; mp 206.5-207.5° C. [alpha]_{D}=+33.9°. The latter was converted to dextrorotatory title product in the manner above in 49% yield; mp 204-205° C.; [alpha]_{D}=+56.9°. Anal. Found: C, 72.94; H, 7.64; N, 4.24.

### EXAMPLE 2

### (1S, 2S)-1-(4-hydroxyphenyl)-2-(4-hydroxy-4-phenylpiperidin-yl)-1-propanol methanesulfonate trihydrate

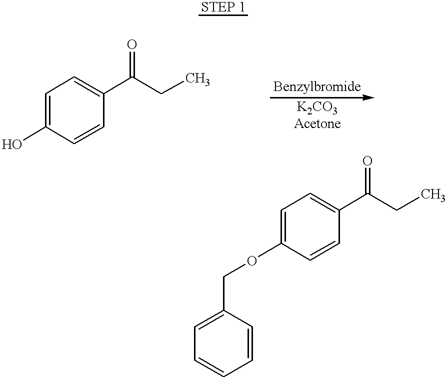

A 50 gallon glass lined reactor was charged with 17.1 gallons of acetone, 8.65 kilograms (kg) (57.7 mol) of 4′-hydroxypropiophenone, 9.95 kg (72.0 mol) of potassium carbonate and 6.8 liters (l) (57.7 mol) of benzylbromide. The mixture was heated to reflux (56° C.) for 20 hours. Analysis of thin layer chromatography (TLC) revealed that the reaction was essentially complete. The suspension was atmospherically concentrated to a volume of 10 gallons and 17.1 gallons of water were charged. The suspension was granulated at 25° C. for 1 hour. The product was filtered on a 30″ Lapp and washed with 4.6 gallons of water followed by a mixture of 6.9 gallons of hexane and 2.3 gallons of isopropanol. After vacuum drying at 45° C., this yielded 13.35 kg (96.4%) of the above-depicted product.

A second run was carried out with 9.8 kg (65.25 mol) of 4′-hydroxypropiophenone using the procedure described above. After drying 15.1 kg (96.3%) of the above-depicted product was obtained.

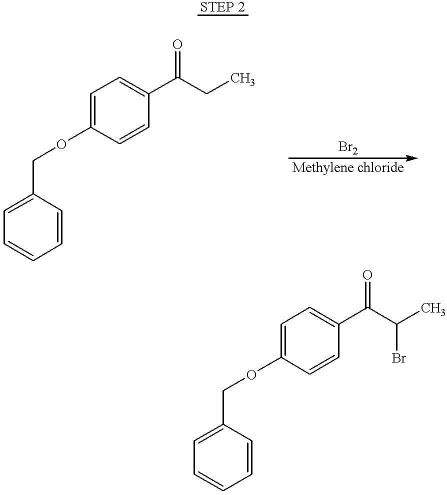

Under a nitrogen atmosphere, a 100 gallon glass lined reactor was charged with 75 gallons of methylene chloride and 28.2 kg (117.5 mol) of the product from step 1. The solution was stirred five minutes and then 18.8 kg of bromine was charged. The reaction was stirred for 0.5 hours at 22° C. Analysis of TLC revealed that the reaction was essentially complete. To the solution was charged 37 gallons of water and the mixture was stirred for 15 minutes. The methylene chloride was separated and washed with 18.5 gallons of saturated aqueous sodium bicarbonate. The methylene chloride was separated, atmospherically concentrated to a volume of 40 gallons and 60 gallons of isopropanol was charged. The concentration was continued until a pot temperature of 80° C. and final volume of 40 gallons were obtained. The suspension was cooled to 20° C. and granulated for 18 hours. The product was filtered on a 30″Lapp and washed with 10 gallons of isopropanol. After vacuum drying at 45° C., this yielded 29.1 kg (77.6%) of the above-depicted product.

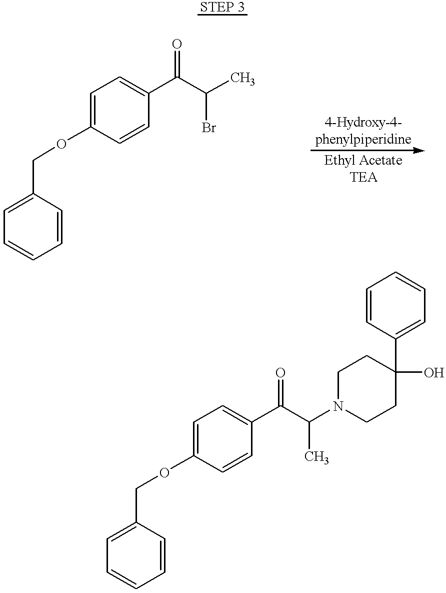

Under a nitrogen atmosphere, a 20 gallon glass lined reactor was charged with 4.90 kg (15.3 mol) of the product from step 2, 7.0 gallons of ethyl acetate, 2.70 kg (15.3 mol) of 4-hydroxy-4-phenylpiperidine and 1.54 kg of triethylamine (15.3 mol). The solution was heated to reflux (77° C.) for 18 hours. The resulting suspension was cooled to 20° C. Analysis by TLC revealed that the reaction was essentially complete. The byproduct (triethylamine hydrobromide salt) was filtered on a 30″ Lapp and washed with 4 gallons of ethyl acetate. The filtrate was concentrated under vacuum to a volume of 17 liters. The concentrate was charged to 48 liters of hexane and the resulting suspension granulated for 2 hours at 20° C. The product was filtered on a 30″Lapp and washed with 4 gallons of hexane. After vacuum drying at 50° C., this yielded 4.9 kg (77%) of the above-depicted product.

A second run was carried out with 3.6 kg (11.3 mol) of the product from step 2 using the procedure described above. After drying 4.1 kg (87%) of the above-depicted product was obtained.

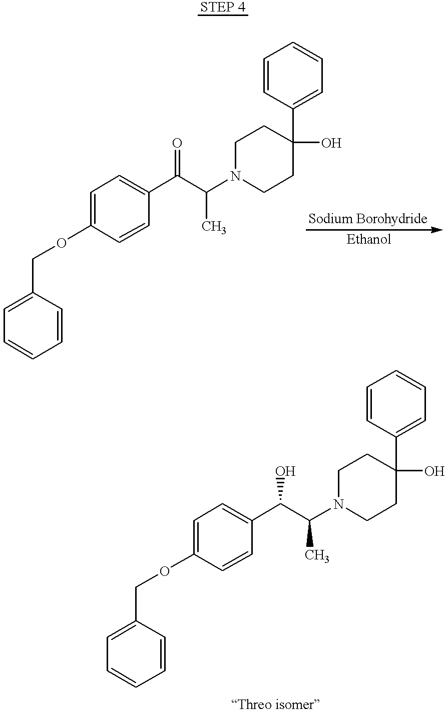

Under a nitrogen atmosphere, a 100 gallon glass lined reactor was charged with 87.0 gallons of 2 B ethanol and 1.7 kg (45.2 mol) of sodium borohydride. The resulting solution was stirred at 25° C. and 9.4 kg (22.6 mol) of the product from step 3 was charged. The suspension was stirred for 18 hours at 25-30° C. Analysis by TLC revealed that the reaction was essentially complete to the desired threo diastereoisomer. To the suspension was charged 7.8 liters of water. The suspension was concentrated under vacuum to a volume of 40 gallons. After granulating for 1 hour, the product was filtered on a 30″Lapp and washed with 2 gallons of 2 B ethanol. The wet product, 9.4 gallons of 2 B-ethanol and 8.7 gallons of water were charged to a 100 gallon glass lined reactor. The suspension was stirred at reflux (78° C.) for 16 hours. The suspension was cooled to 25° C., filtered on 30″ Lapp and washed with 7 gallons of water followed by 4 gallons of 2 B ethanol. After air drying at 50° C., this yielded 8.2 kg (86.5%) of the above-depicted product. This material was recrystallized in the following manner.

A 100 gallon glass lined reactor was charged with 7.9 kg (18.9 mol) of the product from step 3, 20 gallons of 2 B ethanol and 4 gallons of acetone. The suspension was heated to 70° C. producing a solution. The solution was concentrated atmospherically to a volume of 15 gallons. The suspension was cooled to 25° C. and granulated for 1 hour. The product was filtered on a 30″ Lapp. The wet product and 11.7 gallons of 2 B ethanol was charged to a 100 gallon glass lined reactor. The suspension was heated to reflux (78° C.) for 18 hours. The suspension was cooled to 25° C., filtered on a 30″ Lapp and washed with 2 gallons of 2 B ethanol. After air drying at 50° C. this yielded 5.6 kg (70.6%) of the above-depicted product.

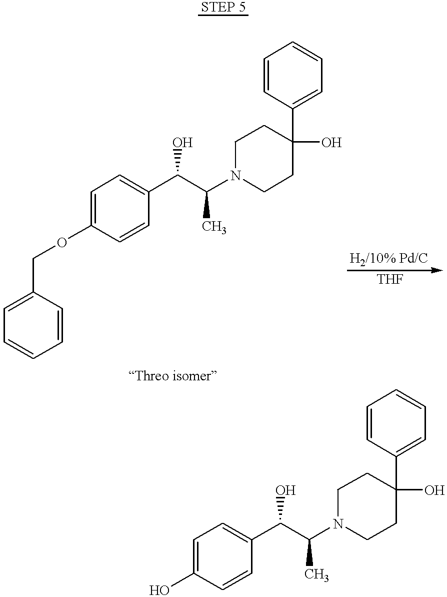

Under a nitrogen atmosphere, a 50 gallon glass lined reactor was charged with 825 g of 10% palladium on carbon (50% water wet), 5.5 kg (13.2 mol) of the product from step 4 and 15.5 gallons of tetrahydrofuran (THF). The mixture was hydrogenated between 40-50° C. for 2 hours. At this time, analysis by TLC revealed that the reduction was essentially complete. The reaction was filtered through a 14″ sparkler precoated with Celite and washed with 8 gallons of THF. The filtrate was transferred to a clean 100 gallon glass lined reactor, vacuum concentrated to a volume of 7 gallons and 21 gallons of ethyl acetate were charged. The suspension was atmospherically concentrated to a volume of 10 gallons and a pot temperature of 72° C. The suspension was cooled to 10° C., filtered on a 30″ Lapp and washed with 2 gallons of ethyl acetate. After air drying at 55° C. this yielded a 3.9 kg (90%) of the above-depicted product (i.e., the free base).

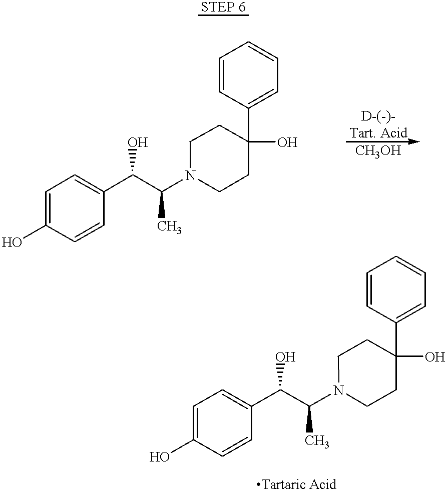

A 100 gallon glass lined reactor was charged with 20 gallons of methanol and 3.7 kg (11.4 mol) of the product from step 5 (i.e., the free base). The suspension was heated to 60° C. and 1.7 kg (11.4 mol) of D-(−)-tartaric acid were charged. The resulting solution was heated to reflux (65° C.) for 3 hours after which a suspension formed. The suspension was cooled to 35° C., filtered on a 30″ Lapp and washed with 1 gallon of methanol. The wet solids were charged to a 100 gallon glass lined reactor with 10 gallons of methanol. The suspension was stirred for 18 hours at 25° C. The suspension was filtered on a 30″ Lapp and washed with 2 gallons of methanol. After air drying at 50° C. this yielded 2.7 kg (101%) of the above-depicted product (i.e., the tartaric acid salt of the free base (R-(+)-enantiomer)). This material was purified in the following manner:

A 100 gallon glass lined reactor was charged with 10.6 gallons of methanol and 2.67 kg (5.6 mol) of the above tartaric acid salt. The suspension was heated to reflux (80° C.) for 18 hours. The suspension was cooled to 30° C., filtered on a 30″ Lapp and washed with 4 gallons of methanol. After air drying at 50° C., this yielded 2.05 kg (76.7%) of the above-depicted product (i.e., the tartaric acid salt of the free base).

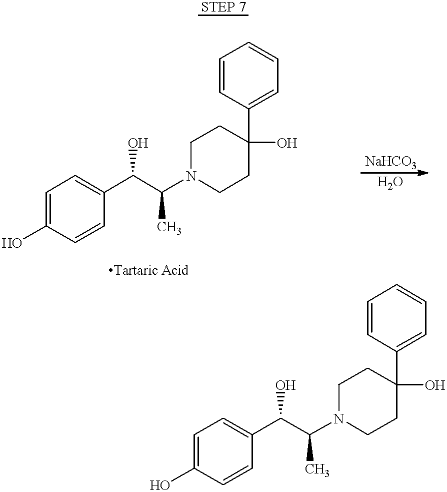

A 55 liter nalgene tub was charged with 30 liters of water and 1056 g (12.6 mol) of sodium bicarbonate at 20° C. To the resulting solution was charged 2.0 kg (4.2 mol) of the product from step 6 (i.e., the tartaric acid salt of the free base). The suspension was stirred for 4 hours during which a great deal foaming occurred. After the foaming ceased, the suspension was filtered on a 32 cm funnel and washed with 1 gallon of water. After air drying at 50° C., this yielded 1.28 kg (93.5%) of the above-depicted product (i.e., the free base).

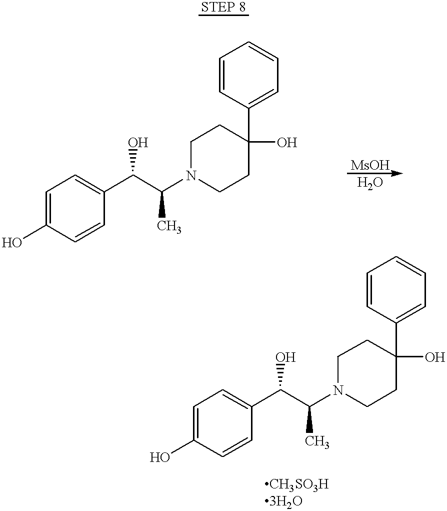

A 22 liter flask was charged with 1277 g (3.9 mol) of product from step 7 and 14 liters of water. The suspension was warmed to 30° C. and 375 g (3.9 mol) of methane sulfonic acid were charged. The resulting solution was warmed to 60° C., clarified by filtering through diatomaceous earth (Celite™) and washed with 2 liters of water. The speck-free filtrate was concentrated under vacuum to a volume of 6 liters. The suspension was cooled to 0-5° C. and granulated for 1 hour. The product was filtered on an 18″ filter funnel and washed with 635 ml of speck-free water. After air drying at 25° C. for 18 hours, this yielded 1646 g (88%) of the above-depicted product (i.e., the mesylate salt trihydrate).

### EXAMPLE 3

### (1R*, 2R*)-1-(4-hydroxy-3-methylphenyl)-2-(4-(4-fluorophenyl)-4-hydroxypiperidin-1-yl)-propan-1-ol-mesylate

A mixture of 3-methyl4-triisopropylsilyloxy-α-bromopropiophenone (9.17 g, 22.97 mmol), 4-(4-fluorophenyl)-4-hydroxypiperidine (6.73 g, 34.45 mmol) and triethylamine (8.0 mL, 57.43 mmol) in in ethanol (180 mL) was refluxed for 6 hours. The solvent was removed at reduced pressure and the residue was partitioned between ethyl acetate and water. The phases were separated and the organic layer was washed with brine, dried over calcium sulfate and concentrated. The residue was flash chromatographed on silica gel (3×3.5 inches packed in hexane) with elution proceeding as follows: 10% ethyl acetate/hexane (1000 mL), nil; 20% ethyl acetate/hexane (700 mL), nil; 20% ethyl acetate/hexane (1300 mL) and 25% ethyl acetate/hexane (600 mL), 7.66 g (65%) of 1-(3-methyl-4-triisopropylsilyloxyphenyl)-2-(4-(4-fluorophenyl)-4-hydroxypiperidin-1-yl)-propan-1-one as a yellow foam which was suitable for use without further purification. A sample recrystallization from ethyl acetate/hexane as white crystals had: m.p. 78-82° C.

A mixture of sodium borohydride (0.564 g, 14.92 mmol) and ethanol (60 mL) was stirred 10 minutes and then 1-(3-methyl-4-triisopropylsilyloxyphenyl)-2-(4-(4-fluorophenyl)-4-hydroxypiperidin-1-yl)-propan-1-one (7.66 g, 14.92 mmol in 10 mL of ethanol) was added with two 30 mL ethanol rinses. The reaction mixture was stirred at ambient temperature overnight. The white solid that precipitated was collected by filtration and dried to yield 5.72 g (74%) of (1R*, 2R*)-1-(3-methyl-4-triisopropylsilyloxyphenyl)-2-(4-(4-fluorophenyl)-4-hydroxypiperidin-1 -yl)-propan-1-ol, which was suitable for use without further purification and had: m.p. 188-189° C.

The product of the above reaction (5.72 g, 11.1 mmol) was dissolved in tetrahydrofuran (150 mL) and tetrabutylammonium fluoride (12.21 mL, 12.21 mmol, 1M tetrahydrofuran solution) was added. The reaction was stirred 1 hour at ambient temperature and then concentrated. The residue was partitioned between ethyl acetate and water and the two phases were separated. The organic layer was slurried with methylene chloride. The white solid that precipitated was collected by filtration and dried to afford 3.41 g (85%) of (1R*, 2R*)-1-(4-hydroxy-3-methylphenyl)-2-(4-(4-fluorophenyl)4-hydroxypipeidin-1-yl)-propan-1-ol. A sample (0.16 g, 0.447 mmol) was converted to the corresponding mesylate salt. The salt was slurried in methanol (8 mL) and methanesulfonic acid (0.029 mL, 0.45 mmol) was added. The mixture was filtered and concentrated. The mixture was then recrystallized from ethanol to give 0.152 g (58%) of the mesylate salt which had: m.p. 215-216° C. Analysis calculated for C₂₁H₂₅FNO₃.CH₄SO₃: C, 58.01; H, 6.64, N, 3.07. Found: C, 57.99; H, 6.72: N, 3.17.

### EXAMPLE 4

### 1R, 2R 1-(4-hydroxy-3-methoxyphenyl)-2-(4-hydroxy-4-phenyl-piperidin-1-yl)-propan-1-ol and 1S, 2S 1-(4-hydroxy-3-methoxyphenyl)-2-(4-hydroxy-4-phenyl-piperidin-1-yl)-propan-1-ol

A mixture of 2-bromo-1-(2,2-diphenyl-benzo(1,3)dioxol-5-yl)-propan-1-one (2.00 g, 4.89 mmol), 4-hydroxy-4-phenylpiperidine (0.9 g, 5.08 mmol) and triethylamine (1.40 mL, 10.04 mmol) in ethanol (50 mL) was refluxed overnight. The solvent was removed at reduced pressure and the residue was partitioned between ether and water. The phases were separated and the organic layer was washed with brine, dried over magnesium sulfate and concentrated. The residue was flash chromatographed on silica gel (2×5 inches packed with hexane) with elution proceeding as follows: 20% ethyl acetate/hexane (500 mL), unweighed forerun; 50% ethyl acetate/hexane (500 mL), 1.76 g (71%) of 1-(2,2)-diphenyl-benzo(1,3)dioxol-5-yl)-2-(4-hydroxy-4-phenylpiperidin-1-yl)-propan-1-one as light tan foam which was suitable for use without further purification and had: NMR δ 7.81 (dd, J=1.7, 8.3 Hz, 1H), 7.70 (d, J=1.6 Hz, 1H), 7.64-7.13 (m, 15H), 6.92 (d, J=8.2 Hz, 1H), 4.07 (q, J=7.0 Hz, 1H), 3.39-3.27 (m, 1H), 2.94-2.59 (m, #H), 2.30-2.04 (m, 2H), 1.74 (brt, J=13.2 Hz, 2H), 1.30 (d, J=6.8 Hz, 3H).

A mixture of sodium borohydride (0.15 g, 3.97 mmol) and ethanol (5 mL) was stirred 10 minutes and then 1-(2,2-diphenyl-benzo(1,3)dioxol-5-yl)-2-(4-hydroxy-4-phenylpiperidin-1-yl)-propan-1-one (1.70 g, 3.36 mmol in 20 mL of ethanol) was added. The reaction was stirred at ambient temperature over the weekend. The white precipitate was collected, rinsed with ethanol and ether and air dried to afford 1.35 g of crude product. The product was recrystallized from ethanol/ethyl acetate/methylene chloride to give 1.05 g (61 %) of 1R*, 2R*)-1-(2,2-diphenyl-benzo(1,3)dioxol-5-yl)-2-(4-hydroxy4-phenylpiperidin-1-yl)-propan-1-ol which had: mp 224-224.5° C. Analysis calculated for C₃₃H₃₃NO₄: C, 78.08; H, 6.55; N, 2.76. Found: C, 78.16; H, 6.46; N, 2.72.

A mixture of the product of the above reaction (1.00 g, 1.97 mmol) and 10% palladium on carbon (0.175 g) in methanol (50 mL) and acetic acid (1.0 mL) was hydrogenated at 50 psi (initial pressure) for 5 hours at ambient temperature. Additional catalyst (0.18 g) was added and the hydrogenation was continued overnight. The reaction was filtered through diatomaceous earth and the filter pad was rinsed with methanol. The filtrate was concentrated and the residue was partitioned between ethyl acetate and saturated aqueous bicarbonate and stirred vigorously for 1 hour. The phases were separated and the aqueous layer was extracted with ethyl acetate (2x). The combined organic layer was washed with water and brine, dried over magnesium sulfate and concentrated The residue was flash chromatographed on silica gel (1×4 inches) with elution proceeding as follows: 20% ethyl acetate/hexane (500 mL), nil; 10% methanol/ethyl acetate (250 mL), 20% methanol/ethyl acetate (250 mL), and 50% methanol/ethyl acetate, 0.51 g (75%) of a light yellow-green solid. The solid was recrystallized from ethanol to afford (1R*, 2R*)-1-(3,4-dihydroxyphenyl)-2-(4-hydroxy-4-phenyl-piperidin-1-yl)-propan-1-ol as a white solid which had: mp 167-168° C. Analysis calculated for C₂₀H₂₅NO₄.0.5 C₂H₆O: C, 68.83; H, 7.70; N, 3.82. Found: C, 68.78; H, 8.05; N, 3.70.

The racemic product was dissolved in ethanol and separated into enantiomers by HPLC using the following chromatographic conditions: Column, Chiralcel OD; mobile phase, 25% ethanol/75% hexane; temperature, ambient (approximately 22° C.); detection, UV at 215 nM. Under these conditions, 1R, 2R 1-(4-hydroxy-3-methoxyphenyl)-2-(4-hydroxy-4-phenyl-piperidin-1-yl) propan-1-ol eluted with a retention time of approximately 9.12 minuntes and 1S, 2S 1-(4-hydroxy-3-methoxyphenyl)-2-(4-hydroxy-4-phenyl-piperidin-1-yl) propan-1-ol eluted with a retention time of approximately 16.26 minutes.

### EXAMPLE 5

### (3R*, 4S)-3-(4-(4-fluorophenyl)-4-hydroxypiperidin-1-yl)-chroman-4,7-diol

A mixture of 7-benzyloxy-3,3-dibromochromanone (54.7 g, 133 mmol), 4-(4-fluorophenyl)-4-hydroxypiperidine (52.0 g, 266 mmol), and triethylamine (38 mL, 270 mmol) in acetonitrile (2.5 L) was stirred 16 hours at ambient temperature. A yellow precipitate formed and was collected, washed well with water and ether, and air dried. The yield of 7-benzyloxy-3-[4-(4-fluorophenyl)-4-hydroxy-pipridine-1-yl]-chromenone was 55.4 g (93%) which was suitable for use without further purification. A sample recrystallized from ethanol/tetrahydrofuran had mp 220-221° C.: NMR DMSO_{∂σ} δ7.99 (d, J=9 Hz, 2H), 7.56-7.40 (m, 8H), 7.18-7.08 (m, 4H), 5.25 (s, 2H), 5.06 (s, 1H), 3.60 (br s, 1H), 3.55-3.35 (m, 1H, partially obscured by water from the NMR solvent), 3.10-2.95 (m, 2H), 2.15-2.00 (m, 2H), 1.71 (br t, J=13.7 Hz, 2H).

Analysis calculated for C₂₇H₂₄FNO₄: C, 72.80; H, 5.43; N, 3.13. Found: C, 72.83; H, 5.82; N, 2.82.

To a slurry of 7-benzyloxy-3-[4-(4-fluorophenyl)-4-hydroxy-piperidine-1-yl] -chromenone (8.24 g, 18.5 mmol) in ethanol (400 mL) and tetrahydrofuran (600 mL) was added sodium borohydride (7.0 g, 185 mmol). The mixture was stirred overnight. Additional sodium borohydride (7.0 g) was added and the reaction mixture was stirred for 3 days. Water was added and the solvent was removed at reduced pressure at 45° C. The solids which formed were collected and washed well with water and then ether. The solid was further dried in vacuo overnight to give 5.01 g, 60% of 3R* 4S* 7-benzyloxy-3-[4-(4-fluorophenyl)-4-hydroxy-piperidin-1-yl]-chroman-4-ol which was suitable for use without further purification. A sample recrystallized from ethyl acetate/chloroform had mp. 194-195° C.; NMR δ7 56-7.30 (m, 8H), 7.06 (long range coupled t, J=8.7 Hz, 2H) 6.63 (dd, J=2.4, 8.5 Hz, 1H), 6.47 (d, J=2.4 Hz, 1H), 5.04 (s, 2H), 4.77 (d, J=4.5 Hz, 1H), 4.37 (dd, J=3.5, 10.4 Hz, 1H), 4.13 (t, J=10.4 Hz, 1H), 3.82 (brs, 1H), 3.11 (br d, J=11.2 Hz, 1H), 2.92-2.71 (m, 4H), 2.21-2.06(m, 2H), 1.87-1.73 (m, 2H), 1.54 (s, 1H).

Analysis calculated for C₂₇H₂₈FNO₄: C, 72.14; H, 6.28; N, 3.12. Found C, 72.15; H, 6.21; N, 3.12.

A mixture of 3R* 4S* 7-benzyloxy-3-[4-(4-fluorophenyl)-4-hydroxy-piperidin-1-yl] -chroman-4-ol (0.80 g, 1.78 mmol), 10% palladium on carbon (0.16 g), methanol (40 mL), and acetic acid (0.8 mL) was hydrogenated for 8 hours with a starting pressure of 48.5 psi. The reaction was filtered through celite and the filtrate was concentrated. The residue was stirred vigorously with ether and saturaturated sodium bicarbonate for 1 hour. The solid was washed with water and ether and dried in vacuo. Recrystallization from ethanol yielded 0.35 g (54%) of 3R* 4S* 3-[4-(4-fluorophenyl)-4-hydroxy-piperidin-1-yl]-chroman-4,7-diol as a white solid which had mp 159-160° C.; NMR DMSO_{∂σ}δ7.55-7.47 (m, 2H), 7.11 (t, J=9 Hz, 2H), 7.02 (d, J=8.4 Hz, 1H)k, 6.32 (dd, J=2.3, 8.3 Hz, 1H), 6.15 (d, J=2.3 Hz 1H), 5.10-4.50 (br m with s at 4.63, 3H), 4.23 (dd, J=2.8, 10.3 Hz, 1H), 4.04 (t, J=10.5 Hz, 1H), 2.99 (br d, J=10.8 Hz, 1H), 2.86 (br d, J=10.7 Hz, 1H), 2.73-2.50 (m, 3H), 2.08-1.90 (m, 2H), 1.58 (br d, J=13 Hz, 2H).

Analysis calculated for C₂₀H₂₂FNO₄.0.25H₂O; C, 66.01; H, 6.23; N, 3.85. Found: C, 66.22; H, 6.58; N. 3.46.

TABLE 1
Affinity of CP-101,606 and other compounds for displacement of the specific binding
of racemic [³H](+)-(1S, 2S)-1-(4-hydroxy-phenyl)-2-(4-hydroxy-4-phenylpiperidino)-1-
propanol or [³H]Prazosin to rat forebrain membranes.

| | | | Ratio |
|---|---|---|---|
| | Racemic | | [³H]Prazosin/ |
| | [³H](+)-(1S, 2S)-1-(4- | | [³H](+)-(1S, 2S)-1-(4- |
| | hydroxy-phenyl)-2-(4- | | hydroxy-phenyl)-2-(4- |
| | hydroxy-4-phenylpiperidino)- | | hydroxy-4- |
| | 1-propanol | [³H]Prazosin | phenylpiperidino)-1- |
| Compound | binding (nM) | binding (nM) | propanol |
| (+)-(1S, 2S)-1- | 13 ± 4 | 19,500 ± 5,000 | 1,500 |
| (4-hydroxy- | | | |
| phenyl)-2-(4- | | | |
| hydroxy-4- | | | |
| phenylpiperidi | | | |
| no)-1-propanol | | | |
| (1R*,2R*)-1- | 14 ± 2.1 | 10,000 | 714 |
| (4-hydroxy-3- | | | |
| methylphenyl)- | | | |
| 2-(4-(4- | | | |
| fluorophenyl)- | | | |
| 4- | | | |
| hydroxypiperid | | | |
| in-1-yl)- | | | |
| propan-1-ol- | | | |
| mesylate | | | |
| (1S, 2S)-1-(4- | 94 | 8100 | 86 |
| hydroxy-3- | | | |
| methoxyphenyl) | | | |
| -2-(4-hydroxy- | | | |
| 4- | | | |
| phenylpiperidin | | | |
| o)-1-propanol | | | |
| (3R,4S)-3-(4- | 18 ± 3 | >10,000 | ≧555 |
| (4- | | | |
| fluorophenyl)- | | | |
| 4- | | | |
| hydroxypiperid | | | |
| in-1-yl)- | | | |
| chroman-4,7- | | | |
| diol | | | |
| Ifenprodil | 70 ± 25 | 114 ± 5 | 1.6 |
| (Comparative) | | | |
| Eliprodil | 450 ± 130 | 980 ± 220 | 2.2 |
| (Comparative) | | | |

1. A method of treating acute, chronic and/or neuropathic pain in a mammal experiencing chronic, acute and/or neuropathic pain comprising administering to said mammal an effective amount of an NR2B selective N-methyl-D-aspartate (NMDA) receptor antagonist having a ratio of NR2B receptor activity to α₁-adrenergic receptor activity of at least about 3:1.

2. The method of claim 1, wherein said NR2B subtype selective NMDA receptor antagonist is a compound of the formula 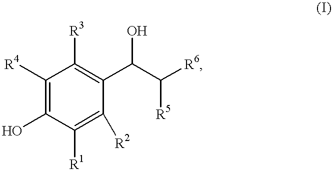
or a pharmaceutically acceptable acid addition salt thereof, wherein:
(a) R² and R⁵ are taken separately and R¹, R², R³ and R⁴ are each independently hydrogen, (C₁-C₆) alkyl, halo, CF₃, OH or OR⁷ and R⁵is methyl or ethyl; or
(b) R² and R⁵ are taken together and are 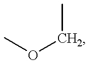
forming a chroman-4-ol ring, and R¹, R³ and R⁴ are each independently hydrogen, (C₁-C₆) alkyl, halo, CF₃, OH or OR⁷;
R⁶ is 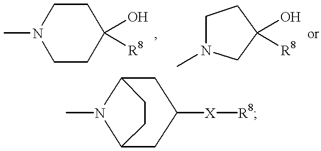
R⁷ is methyl, ethyl, isopropyl or n-propyl;
R⁸ is phenyl optionally substituted with up to three substituents independently selected from the group consisting of (C₁-C₆) alkyl, halo and CF₃;
X is O, S or (CH₂)ₙ; and
n is 0, 1, 2, or 3.

3. The method of claim 2, wherein said compound is (+)-(1S, 2S)-1-(4-hydroxy-phenyl)-2-(4-hydroxy-4-phenylpiperidino)-1-propanol or a pharmaceutically acceptable acid addition salt thereof.

4. The method of claim 2, wherein said compound is (1S, 2S)-1-(4-hydroxy-3-methoxyphenyl)-2-(4-hydroxy-4-phenylpiperidino)-1-propanol or a pharmaceutically acceptable acid addition salt thereof.

5. The method of claim 2, wherein said compound is (3R,4S)-3-(4-(4-fluorophenyl)-4-hydroxypiperidin-1-yl)-chroman-4,7-diol or a pharmaceutically acceptable acid addition salt thereof.

6. The method of claim 2, wherein said compound is (1R*,2R*)-1-(4-hydroxy-3-methylphenyl)-2-(4-(4-fluorophenyl)-4-hydroxypiperidin-1-yl)-propan-1-ol-mesylate

7. The method of claim 1, wherein said ratio of NR2B receptor activity to α₁-adrenergic receptor activity is at least about 5:1.
